SEQUENCE LISTING (2 sequences)

SEQ ID NO: 1 - DNA, 492 bp, Homo sapiens
  modified_base (4): a, c, t, g, unknown or other
gccngatctcctttatcagccctcactccttctctaggcgccggaattcggcacgagggttggactgtgaagaaacatggcggccgcgacgttgacttcgaaattgtactccctgctgttccgcaggacctccaccttcgccctcaccatcatcgtgggcgtcatgttcttcgagcgcgccttcgatcaaggcgcggacgctatctacgaccacatcaacgaggggaagctgtggaaacacatcaagcacaagtatgagaacaagtagttccttggaggcccccatccaggccagaaggaccaggtccacccagcagctgtttgcccagagctggagcctcagcttgaagatgatgctcaaggtactcttcatggaccaccattcgctgttggcaagaaacggttttacttacaaaacagactctttaccttctgctgtgtttgaagtatgtttagtcagcatgctcaggaaataaatgtgaattgcccttg

SEQ ID NO: 2 - DNA, 840 bp, Homo sapiens
  modified_base (685): a, c, t, g, unknown or other
cttcctctaggcgccggaattcggcacgagggccgaaaggaaagaaggccaagggaaagaaggtggctccggccccagctgtcgtgaagaagcaggaggctaagaaagtggtgaatcccctgtttgagaaaaggcctaagaattttggcattggacaggacatccagcccaaaagagacctcacccgctttgtgaaatggccccgctatatcaggttgcagcggcagagagccatcctctataagcggctgaaagtgcctcctgcgattaaccagttcacccaggccctggaccgccaaacagctactcagctgcttaagctggcccacaagtacagaccagagacaaagcaagagaagaagcagagactgttggcccgggccgagaagaaggctgctggcaaaggggacgtcccaacgaagagaccacctgtccttcgagcaggagttaacaccgtcaccaccttggtggagaacaagaaagctcagctggtggtgattgcacacgacgtggatcccatcgagctggttgtcttcttgcctgccctgtgtcgtaaaatgggggtcccttactgcattatcaagggaaaggcaagactgggacgtctagtccacaggaagacctgcaccactgtcgccttcacacaggtgaactcggaaaacaaaggcgctttggctaagctggnggaagctatcaggaccanttacaatgacagatacgatgagatccgccgtcactggggtggcaatgtcctggtcctaagtctgnggctcgtatcgccagctcgaaaggcaaggctaaaaacttgccctaactgggtaaannncctgtngantttct

(19) United States
(12) Patent Application Publication
(10) Pub. No.: US 2008/0145369 A1
(43) Pub. Date: Jun. 19, 2008

(54) Autocrine Growth Factor Receptors and Methods

(76) Inventor: Ginette Serrero, Ellicott City, MD (US)

Correspondence Address: DICKSTEIN SHAPIRO LLP, 1825 EYE STREET NW, Washington, DC, 20006-5403, US

(21) Appl. No.: 11/665,359

(22) PCT Filed: Oct. 12, 2005
(86) PCT No.: PCT/US05/36876

§ 371 Date: Oct. 19, 2007

Related U.S. Application Data
(60) Provisional application No. 60/617,683, filed on Oct. 13, 2004.

(51) Int. Cl. A61K 39/395; G01N 33/574; C12Q 1/68; C07H 21/00; C07K 14/475; A61K 31/7088; A61K 38/18

(52) U.S. Cl. 424/158.1; 435/7.23; 435/6; 514/2; 514/44; 536/23.1; 530/399

(57) ABSTRACT

Embodiments of the invention provide methods and compositions for method for diagnosing and treating diseases including cancer. In one embodiment diagnosing tumorigenicity is accomplished by measuring the level of PCDGF receptor expression in a tissue sample suspected of being tumorigenic. In another embodiment, a determination of whether tumorigenic cells will be responsive to an anti-PCDGF therapy is accomplished by determining whether there is a measured level of PCDGF receptor in a tissue sample suspected of being tumorigenic. In yet another embodiment, a method for treating a subject having a disease related to the amplification or overexpression of PCDGF includes administering a PCDGF receptor or fragment thereof to a patient in amounts effective to inhibit the biological activity of PCDGF. The invention also provides isolated PCDGF receptor nucleic acid molecules and proteins encoded by the PCDGF receptor nucleic acid molecules.

### CROSS REFERENCE TO RELATED APPLICATION

The present application claims priority to U.S. Application Ser. No. 60/617,683, filed on Oct. 13, 2004, the disclosure of which is herewith incorporated by reference in its entirety.

### FIELD OF THE INVENTION

This invention relates to cell biology, physiology and medicine, and concerns an 88 kDa glycoprotein growth factor (“GP88”) and compositions and methods which affect the expression and biological activity of GP88. This invention also relates to kit products, compositions and methods which are useful for diagnosis and treatment of diseases including cancer.

### BACKGROUND

The proliferation and differentiation of cells in multicellular organisms is subject to a highly regulated process. A distinguishing feature of cancer cells is the absence of control over this process; proliferation and differentiation become deregulated resulting in uncontrolled growth. Significant research efforts have been directed toward better understanding this difference between normal and tumor cells. One area of research focus is growth factors and, more specifically, autocrine growth stimulation.

Growth factors are polypeptides which carry messages to cells concerning growth, differentiation, migration and gene expression. Typically, growth factors are produced in one cell and act on another cell to stimulate proliferation. However, certain malignant cells, in culture, demonstrate a greater or absolute reliance on an autocrine growth mechanism. Malignant cells which observe this autocrine behavior circumvent the regulation of growth factor production by other cells and are therefore unregulated in their growth.

Study of autocrine growth control advances understanding of cell growth mechanisms and leads to important advances in the diagnosis and treatment of cancer. Toward this end, a number of growth factors have been studied, including insulin-like growth factors (“IGF1” and “IGF2”), gastrin-releasing peptide (“GRP”), transforming growth factors alpha and beta (“TGF-a” and “TGF-b”), and epidermal growth factor (“EGF”).

The present invention is directed to a recently discovered growth factor. This growth factor was first discovered in the culture medium of highly tumorigenic “PC cells,” an insulin-independent variant isolated from the teratoma derived adipogenic cell line 1246. This PC-cell-derived growth factor (“PCDGF” or “GP88”) is an 88-kDa glycoprotein autocrine growth factor expressed in a tightly regulated fashion in normal cells but overexpressed and unregulated in tumorigenic cells. Inhibition of PCDGF expression or activity inhibits the growth of tumorigenic cells. PCDGF is composed of a 68-KDa protein core and a 20-KDa carbohydrate moiety. PCDGF belongs to a novel family of double cysteine rich polypeptides and was originally isolated from the culture medium of the highly tumorigenic mouse teratoma-derived cell line PC. PCDGF has been shown to be overexpressed in several mouse and human tumors including liver, kidney, breast, bone, bone marrow, testes, prostate, brain, ovary, skin, and lung.

What is needed are methods and compositions for using a receptor for PCDGF to interfere with the biological activity of PCDGF and to diagnose and treat diseases such as cancer.

### BRIEF DESCRIPTION OF THE DRAWINGS

FIG. 1 shows the sequence of the PCDGF receptor isolated from 6G8 expression cloned cells.

FIG. 2 shows the cDNA sequence of the PCDGF receptor isolated from cells that bind to PCDGF.

FIG. 3 shows the results of a Northern blot analysis indicating that there is no hybridization with mRNA from 3C3.

FIG. 4 shows in vivo GP88 expression levels in C3H mice tumor tissues and in surrounding normal tissues.

FIG. 5 shows a cDNA library from AG-BC1 cells constructed into pLXSN retroviral vector that contains a neomycin resistance gene for selection of cells.

FIG. 6 illustrates an exemplary process for the expression cloning of PCDGF receptor.

FIG. 7 is a chart depicting the ability of cell lines screened using PCDGF receptor antibody 6G8 for the acquisition of ability to bind PCDGF.

FIG. 8 shows the immunohistochemical staining of cells with biotinylated 6G8.

FIG. 9 shows Western blot analysis results of phosphorylated Erk1/2 (p44/42). The results show that 2F6 cells, expression cloned by 6G8 selection, bind to the 6G8 antibody and respond to PCDGF as indicated by stimulating MAP kinase activity.

FIG. 10 shows the binding of biotinylated PCDGF to expression cloned cells 4 B4, V-1P.

FIG. 11 shows the isolation of genomic DNA from 2F6.

FIG. 12 shows the isolation of a cDNA insert contained in the PCDGF select cells V-1P genomic DNA using retroviral pLXSN primers to amplify the flanking cDNA.

### SUMMARY

Embodiments of the invention provide methods and compositions for method for diagnosing and treating diseases including cancer. In one embodiment diagnosing tumorigenicity is accomplished by measuring the level of PCDGF receptor expression, for example, in a tissue sample suspected of being tumorigenic. In another embodiment, a determination of whether tumorigenic cells will be responsive to an anti-PCDGF therapy is accomplished by determining whether there is a measured level of PCDGF receptor, for example, in a tissue sample suspected of being tumorigenic. The term “tissue” refers to any tissue or fluid in a human or animal including, but not limited to breast, prostate, blood, serum, cerebrospinal fluid, liver, kidney, breast, head and neck, pharynx, thyroid, pancreas, stomach, colon, colorectal, uterus, cervix, bone, bone marrow, testes, brain, neural tissue, ovary, skin, and lung

In yet another embodiment, a method for treating a subject having a disease related to the amplification or overexpression of PCDGF includes administering a PCDGF receptor or fragment thereof to a patient in amounts effective to inhibit the biological activity of PCDGF. Embodiments of the invention also include an isolated nucleic acid molecule having SEQ ID NO:1 and fragments thereof; an isolated nucleic acid molecule having SEQ ID NO:2 and fragments thereof; an isolated protein encoded by the nucleic acid molecule having SEQ ID NO:1; and an isolated protein encoded by the nucleic acid molecule having SEQ ID NO:2.

The accompanying drawings, which are incorporated in and constitute a part of this specification, illustrate embodiments of the invention and, together with the description, serve to explain the principles of the invention.

### DETAILED DESCRIPTION

Reference will now be made in detail to the presently preferred embodiments of the invention, which, together with the following examples, serve to explain the principles of the invention.

PCDGF is a growth modulator for a variety of cell lines, including fibroblasts, PC cells, and mammary epithelial cells. Comparison of the expression of PCDGF in the highly tumorigenic PC cells and in parent 1246 cells demonstrated that PCDGF expression was very low in the non-tumorigenic cells and was overexpressed in the highly tumorigenic cells. The same result was observed in human breast carcinomas where PCDGF expression was very low in non-tumorigenic mammary epithelial cells and increased in breast carcinoma cells.

PCDGF antagonists (e.g., anti-PCDGF antibodies, antisense nucleic acids, small-inhibitory RNA (siRNA)) inhibit or interfere with the activity of PCDGF and with the growth of tumorigenic cells. In both teratoma-derived cells and breast cancer cells, PCDGF activity was inhibited by treating the cells with an anti-PCDGF neutralizing antibody or by transfecting the cells with an antisense PCDGF cDNA. Treatment of cells with PCDGF antagonists in teratoma cells or breast carcinoma cells completely inhibited cell proliferation and tumorigenesis in vivo.

Scatchard analysis of binding of ¹²⁵I-PCDGF to the mink lung epithelial cell line CCL64 revealed the presence of two classes of cell surface receptors: a high affinity class with a Kd of 4.3+/−1.5×10⁻¹¹ M and 560+/−170 sites/cell, and a low affinity class of receptors with a Kd of 3.9+/−1.9×10⁻⁹ M and 17,000+/−5900 sites/cell. Cross-linking studies and autoradiographic analysis revealed the presence of one major cross-linked band with a molecular weight of 190-195 KDa, corresponding to a molecular weight for the unbound receptor of about 110 KDa for the major band. PCDGF receptors belong to the tyrosine kinase family of receptors. Upon binding of PCDGF to the cell surface, the PCDGF receptor is activated by phosphorylation on tyrosine residues, resulting in phosphorylation of several signaling molecules, including IRS-1, SHC, and Grb2, and leading to activation of MAP kinase ERK-2.

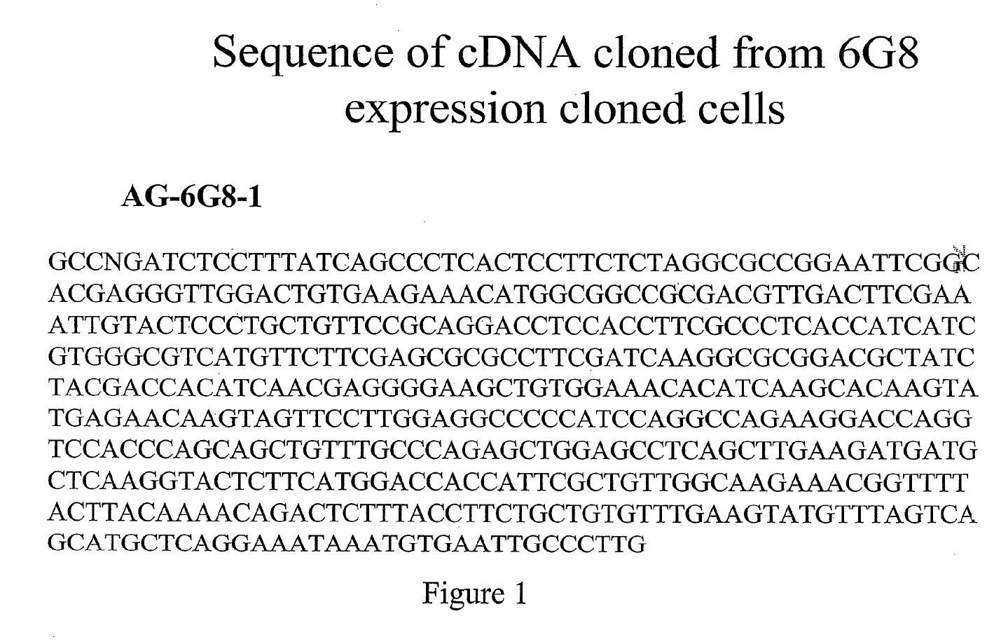

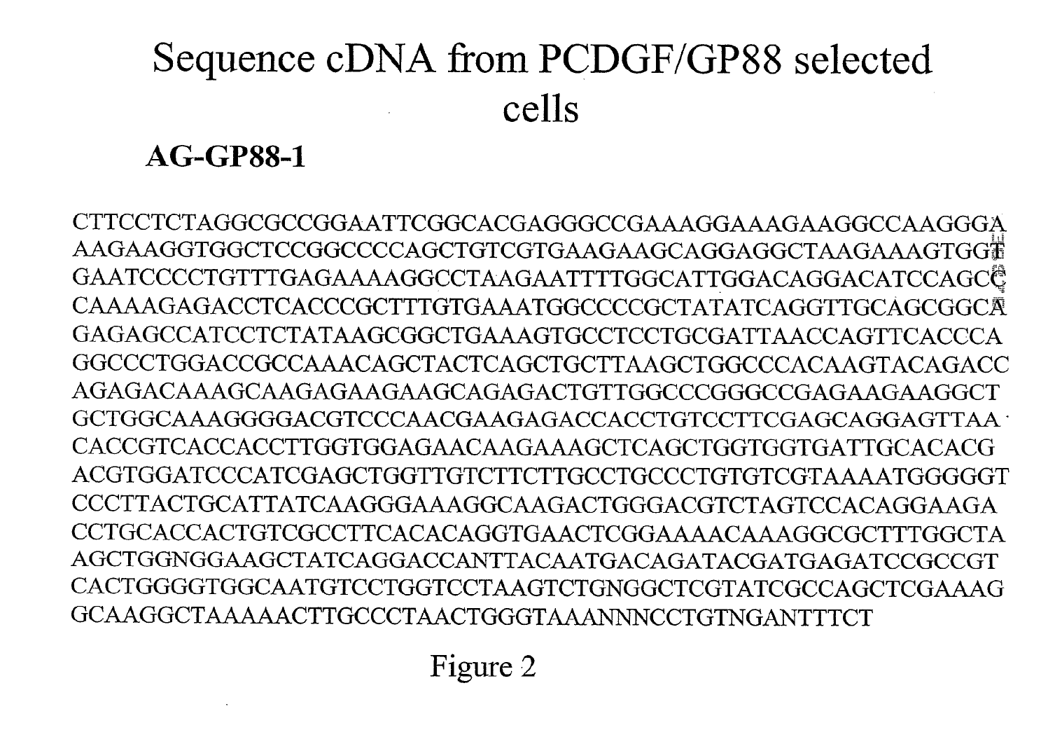

Preferred embodiments of the invention include isolated nucleic acid molecules encoding the PCDGF receptor. The term “receptor” includes co-receptors that modulates ligand binding or the consequences of ligand binding. This interaction may result in the formation of a multi-component complex with a number of possible effects. The receptor/co-receptor complex may stabilize or enhance ligand binding or it may initiate or enhance the signaling and/or functional response to ligand binding. In one embodiment, the nucleic acid molecules encoding the PCDGF receptor comprise the nucleic acid sequence shown in FIG. 1 (SEQ ID NO:1) and/or the nucleic acid sequence shown in FIG. 2 (SEQ ID NO:2), and fragments thereof. Other preferred embodiments of the invention include proteins encoded by the PCDGF receptor nucleic acid molecules (e.g., SEQ ID NO:1 and SEQ ID NO: 2). In another embodiment, the nucleic acid molecules encoding the PCDGF receptor consist essentially of the nucleic acid sequence shown in FIG. 1 (SEQ ID NO:1) and/or the nucleic acid sequence shown in FIG. 2 (SEQ ID NO:2), and fragments thereof. The term “consist essentially of” refers to the nucleic acid molecules shown in FIG. 1 (SEQ ID NO:1) and/or the nucleic acid molecules shown in FIG. 2 (SEQ ID NO:2) and additional components those that do not materially affect the basic and novel characteristics of the claimed invention (e.g., carriers, buffers, chemical moieties, and toxins).

Elevated levels of PCDGF receptor nucleic acid molecules and/or PCDGF receptor proteins are associated with the initiation, development, and progression of a variety of cancers (e.g., teratoma, neuroblastoma, glioblastoma, astrocytoma, sarcomas, and cancers of the, among others, breast, prostate, blood, cerebrospinal fluid, liver, kidney, breast, head and neck, pharynx, thyroid, pancreas, stomach, colon, colorectal, uterus, cervix, bone, bone marrow, testes, brain, neural tissue, ovary, skin, and lung). An increase in tumorigenic properties is associated with an increase in PCDGF receptor expression and/or an increase in PCDGF responsiveness. The level of expression of PCDGF receptors in tumor tissue is greater than in surrounding normal tissues.

Accordingly, increase of PCDGF receptor expression levels can be used as a diagnostic approach to detecting tumors and cancer (e.g., teratoma, neuroblastoma, glioblastoma, astrocytoma, sarcomas, and cancers of the breast, prostate, blood, cerebrospinal fluid, liver, kidney, breast, head and neck, pharynx, thyroid, pancreas, stomach, colon, colorectal, uterus, cervix, bone, bone marrow, testes, brain, neural tissue, ovary, skin, and lung). In human tumor biopsies, a change (e.g., increase) in PCDGF receptor expression when compared to the level of PCDGF receptor in normal corresponding tissues is indicative of the state of tumorigenicity or malignancy of the tissue biopsy analyzed. Increased expression of PCDGF receptors can be measured, for example, at the mRNA level or at the protein level. For example, PCDGF receptor mRNA expression can be measured either by Northern blot analysis, RNAse protection assay or RT-PCR. PCDGF receptor protein expression can be quantified, for example, by ELISA, EIA or RIA using an anti-PCDGF antibody. The level of PCDGF receptor expression in tissue extracts in comparison to corresponding normal tissues can be used to predict the degree of tumorigenicity of a particular cancer or to determine whether this particular cancer will be responsive to anti-PCDGF therapy. In one embodiment, cells having about a 10-fold increase in expression of the PCDGF receptor are highly tumorigenic. In another embodiment of the invention, breast cells having a 10 fold increase in the level of the PCDGF receptor are highly tumorigenic and resistant to the antineoplastic effects of antiestrogen therapy.

The term “theranostics” refers to the use of diagnostic testing to diagnose disease, choose the correct treatment regime, and monitor the patient's response to therapy. Measuring the levels of PCDGF receptors can be used for theranostics related to detection, treatment, and monitoring of tumors and cancers (e.g., teratoma, neuroblastoma, glioblastoma, astrocytoma, sarcomas, and cancers of the breast, prostate, blood, cerebrospinal fluid, liver, kidney, breast, head and neck, pharynx, thyroid, pancreas, stomach, colon, colorectal, uterus, cervix, bone, bone marrow, testes, brain, neural tissue, ovary, skin, and lung). For example, elevated levels of PCDGF receptors in a biopsy or tissue sample compared to normal tissue indicates tumorigenicity and/or the presence of a tumor or cancer. A PCDGF antagonist (e.g., PCDGF antibody, PCDGF receptor, PCDGF antisense, and PCDGF siRNA) and/or another anti-tumor drug can be used to treat the tumor. The tumorigenicity of the tumor or cancer can be monitored by periodically measuring the level of the PCDGF receptor. In one embodiment of the invention, the level of PCDGF receptor in blood is indicative of the tumorigenicity of a patient's tumor or cancer.

In another preferred embodiment, the invention is directed to cells and cell lines capable of producing PCDGF receptors (e.g., V′1-P, 2F6, AG-BC-1 or O4EM). Isolation and cloning of these cells lines are described below. Cell lines that produce PCDGF receptors can be used to generate PCDGF receptor nucleic acids and proteins and for the development of PCDGF receptor antibodies, antisense molecules and siRNA.

In another preferred embodiment, PCDGF receptor nucleic acids and proteins can be used to inhibit the biological activity of PCDGF. For example, PCDGF receptor proteins can be administered to a patient to interfere with or block the interaction of PCDGF with endogenous PCDGF receptor on the surface of a tumor cell. In this embodiment, the administered PCDGF receptor antagonizes the biological activity of PCDGF and can be used to inhibit or interfere with tumor cell growth. In another embodiment, biologically active fragments of PCDGF receptors (e.g., fragments that retain the ability to bind to PCDGF) can be used to inhibit or interfere with tumor cell growth.

PCDGF receptors and fragments thereof can be used to treat diseases related to the amplification or overexpression of PCDGF including, but not limited to, cancer, HIV and other viral infections, autoimmune diseases, and inflammation. PCDGF receptors and fragments thereof can also be used to prevent the occurrence or recurrence of cancer or a tumor by administering the PCDGF receptor and/or fragment to a patient in need of treatment. PCDGF receptors and fragments can be included in pharmaceutical compositions for administration to a patient.

Antagonists to PCDGF receptors (antibodies, antisense, siRNA, and small molecules) can be used to inhibit or suppress the activity of PCDGF and diseases related to amplification and overexpression of PCDGF. The term “PCDGF receptor antagonist” refers to any molecule (e.g., protein, antibody, peptide, small molecule, nucleic acid, antisense, or siRNA) that is capable of binding, interfering with, or inhibiting the activity of the PCDGF receptor or any analogs or derivatives of the PCDGF receptor that retain the properties of the PCDGF receptor.

In one embodiment, a PCDGF receptor antagonist includes a molecule that can target or selectively bind to the PCDGF receptor and, for example, deliver a toxin or other compound or molecule to kill a cell or inhibit cell growth. For example, a PCDGF receptor antibody can be coupled to a toxin or chemotherapeutic agent that is delivered to a tumor cell after the antibody binds to the PCDGF receptor. PCDGF receptor antagonists also include molecules (e.g., peptides, small molecules, antisense molecules, and siRNA) that modulate the biological activity of molecules that regulate the activity of the PCDGF receptor. A PCDGF antagonist can be an antibody that recruits an immune response, e.g., through ADCC (antibody dependent cell cytotoxicity).

PCDGF receptor antagonists also include antibodies that immunospecifically bind a PCDGF receptor and block the binding of PCDGF to the receptor. Such anti-PCDGF receptor antibodies include antibodies produced from hybridoma cell lines including, but not limited to, 6G8 hybridoma cell line (ATCC Accession Number PTA-5263) and 5A8 hybridoma cell line (ATCC Accession Number PTA-5594).

The term antibody used herein refers to an antibody or an antigen-binding fragment thereof that immunospecifically binds to the PCDGF receptor. In other words, an immunospecific antibody is specific for its antigen target (e.g., does not non-specifically bind to or associate with other antigens). Preferably such antibodies do not cross-react with other antigens. These specific antibodies include but are not limited to human and non-human polyclonal antibodies, human and non-human monoclonal antibodies (mAbs), chimeric antibodies, anti-idiotypic antibodies (anti-IdAb), neutralizing antibodies, non-neutralizing antibodies, and humanized antibodies and fragments thereof.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see e.g., Wu and Wu, 1987 J. Biol. Chem., 262: 4429 4432). Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In a preferred embodiment, it may be desirable to introduce the pharmaceutical compositions of the invention into the affected tissues by any suitable route. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g. in conjunction with a wound dressing after surgery, by injection, by means of a catheter, by means of a suppository, or by means of an implant, said implant being of a porous, non porous, or gelatinous material, including membranes, such as sialastic membranes, or fibers. In one embodiment, administration can be by direct injection at the site (or former site) of diseased tissues.

In another embodiment, the pharmaceutical composition can be delivered in a vesicle, in particular a liposome (see e.g., Langer, 1990 Science 249: 1527-1533; Treat et al., in Liposomes in the Therapy of Infectious Disease and Cancer, Lopez Berestein and Fidler (eds.), Liss, New York, pp. 353-365 (1989); Lopez-Berestein, ibid., pp. 317-327; see generally ibid.).

In yet another embodiment, the pharmaceutical composition can be delivered in a controlled release system. In one embodiment, a pump may be used (see Langer, supra; Sefton, 1987 CRC Crit. Ref. Biomed. Eng. 14: 201; Buchwald et al., 1980 Surgery 88:507; and Saudek et al., 1989 N. Engl. J. Med. 321: 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983 J. Macroinol. Sci. Rev. Macromol. Chem. 23: 61; see also Levy et al., 1985 Science 228:190; During et al., 1989 Ann. Neurol. 25:351; Howard et al., 1989, J. Neurosurg. 71:105). In yet another embodiment, a controlled release system can be placed in proximity of the composition's target, i.e., the breast tissue, thus requiring only a fraction of the systemic dose (see e.g., Goodson, in Medical Applications of Controlled Release, supra, vol. 2, pp. 115-138 (1984)). Other controlled release systems are discussed in the review by Langer (1990, Science 249:1527-1533).

The pharmaceutical compositions of the present invention further comprises a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2 ethylamino ethanol, histidine, procaine, etc.

The amount of the pharmaceutical composition of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose response curves derived from in vitro or animal model test systems. Suppositories generally contain active ingredient in the range of 0.5% to 10% by weight; oral formulations preferably contain 10% to 95% active ingredient.

It is to be understood that application of the teachings of the present invention to a specific problem or environment will be within the capability of one having ordinary skill in the art in light of the teachings contained herein. The present invention is more fully illustrated by the following non-limiting example.

### EXAMPLES

PCDGF receptors can be identified and isolated by transfecting cells which do not express PCDGF receptors with nucleic acid clones containing putative PCDGF receptor nucleic acids obtained, for example, from a cDNA expression library. The resulting transfected cells can be screened for the acquisition of the ability to bind PCDGF or to bind an antibody to PCDGF receptor. We have shown that the Chinese Hamster Ovary cell line CHO does not bind or respond to PCDGF. Thus, CHO cells can be used as recipient cells in the expression cloning strategy.

FIG. 1 shows the sequence of the PCDGF receptor isolated from 6G8 expression cloned cells. FIG. 2 shows the cDNA sequence of the PCDGF receptor isolated from cells that bind to PCDGF. Northern blot analysis was performed to examine the size of the mRNA that hybridizes to the PCDGF receptor expression cell lines.

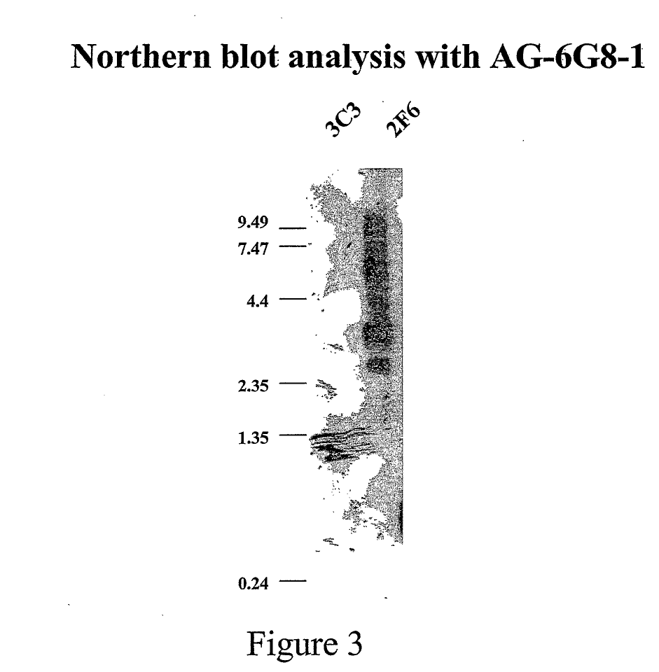

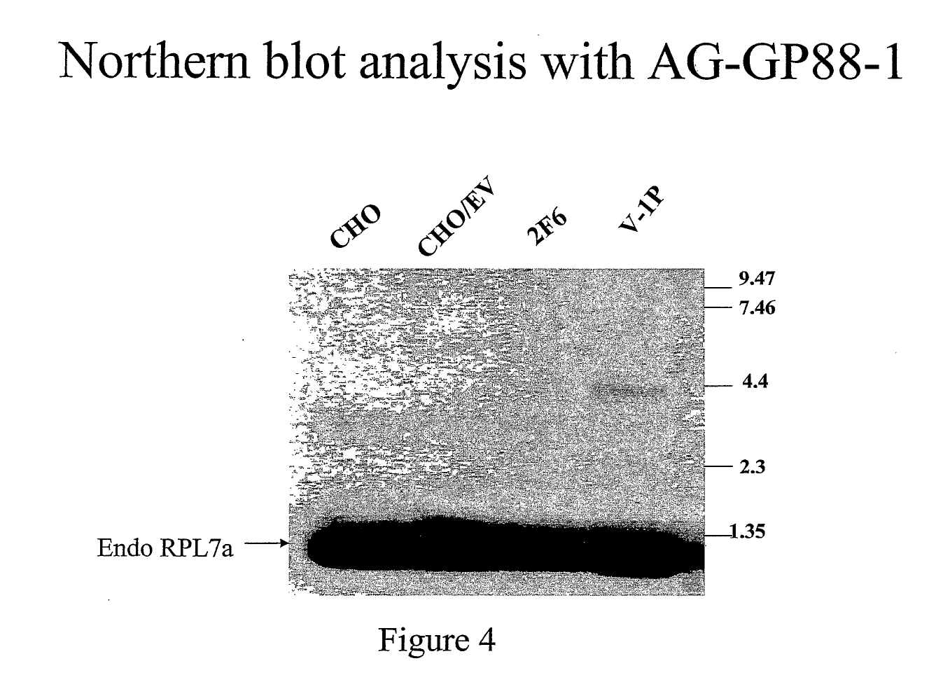

As shown in FIG. 3, Northern blot analysis indicate that there is no hybridization with mRNA from 3C3 whereas several species of mRNA hybridizes with 2F6 mRNA indicating either several messages or several transcriptional start sites. mRNA was extracted from 2F6 cells (cell line that binds 6G8). 3C3 is a cell line isolated from the expression cloning system but does not bind 6G8 or PCDGF. 3C3 was used as a negative control. As shown in FIG. 4, Northern blot analysis was carried out to investigate the size of the mRNA that hybridized to the labeled AGGP88-1 probe. One hybridization band with a size of about 1 kB was observed in all cell lines. This corresponds to the endogenous ribosomal protein RPL 7a mRNA seen in all cells lines as it is conserved throughout species. The second band with a size of about 4 kB is only observed in the V1-P cells from which AG-GP88-1 cDNA was isolated. This band corresponds to the PCDGF receptor mRNA.

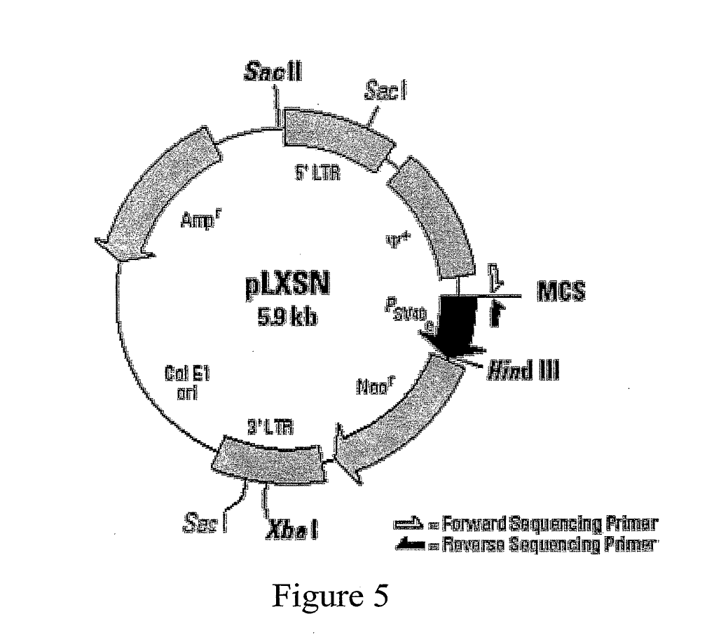

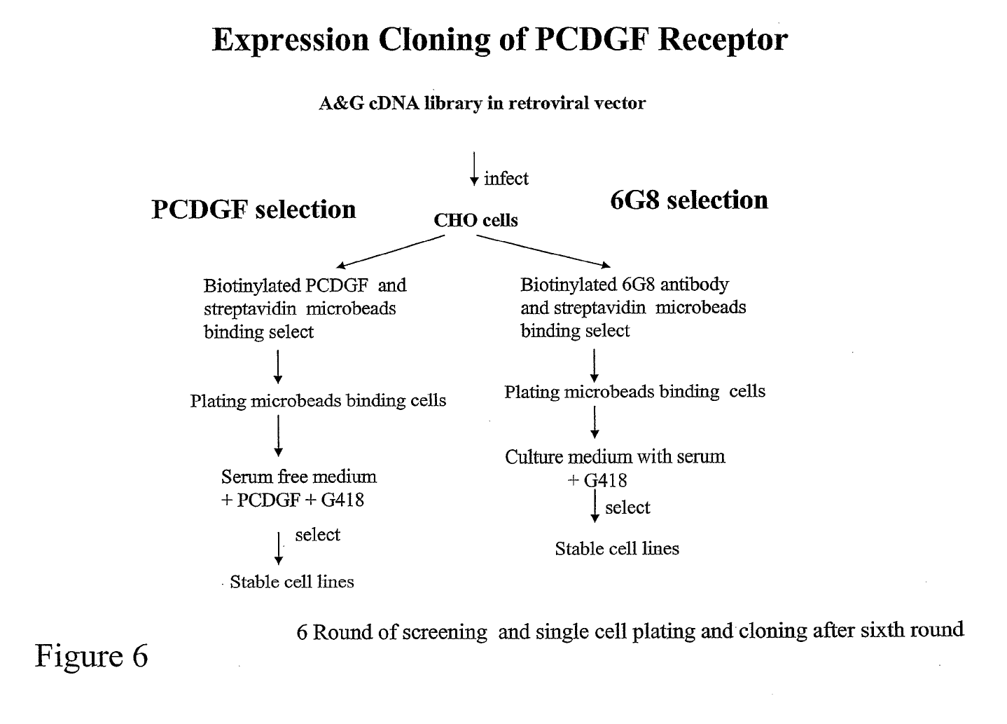

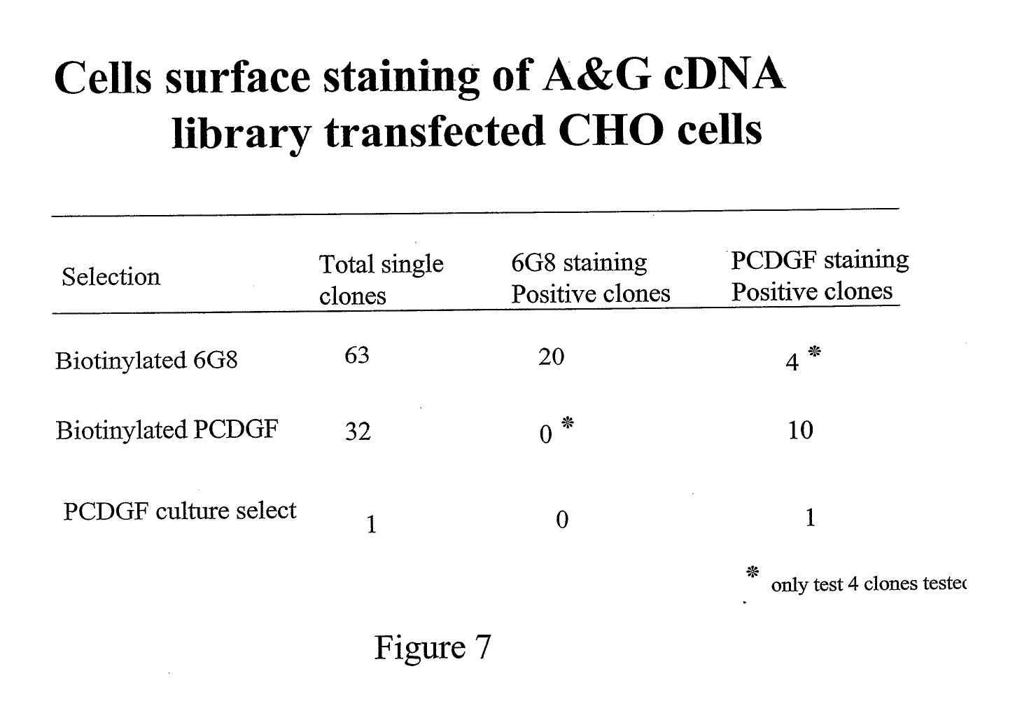

A cDNA library from AG-BC1 cells was constructed into pLXSN retroviral vector that contains a neomycin resistance gene for selection of cells (FIG. 5). Once the library constructed, the cloned cell lines were screened using PCDGF receptor antibody 6G8 for the acquisition of ability to bind PCDGF. Cell lines were also isolated based on the cell line's ability to bind PCDGF. (FIG. 6). As shown in FIG. 7, out of the twenty cell line clones capable of binding 6G8, four were also able to bind to PCDGF.

We have developed a breast cancer cell line that overexpresses the PCDGF receptor and established a cDNA library from these cells that can be used as the source of PCDGF/PCDGF Receptor cDNA. A breast cancer retroviral cDNA library was constructed using mRNA from a breast cancer cell line originally called O4EM, now renamed AG-BC1 that overexpresses PCDGF/PCDGF receptor (named thereafter PCDGF-R). AG-BC1 showed a 10 fold increase in PCDGF/PCDGF binding when compared to breast cancer MCF-7 cells indicating that these cells overexpress PCDGF-R.

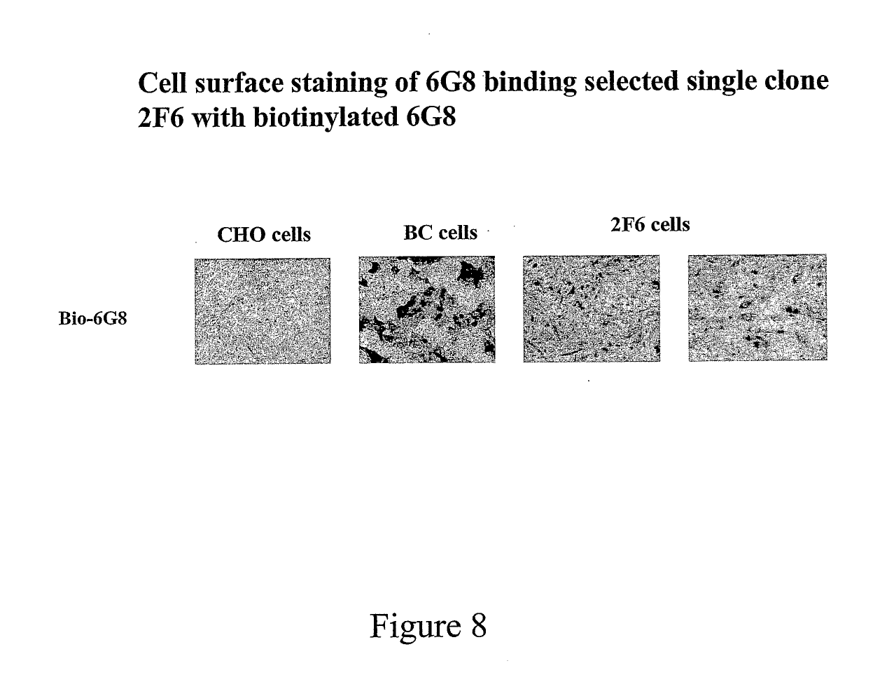

Staining of cells with biotinylated 6G8 followed by streptavidin—HRP followed by chemical detection is shown in FIG. 8. CHO cells do not bind to or express PCDGF and are used as a negative control. MCF-7 and BC cells are positive control cells which express and bind to PCDGF. 2F6 is a positive clone identified during the screening procedure and produces PCDGF receptor.

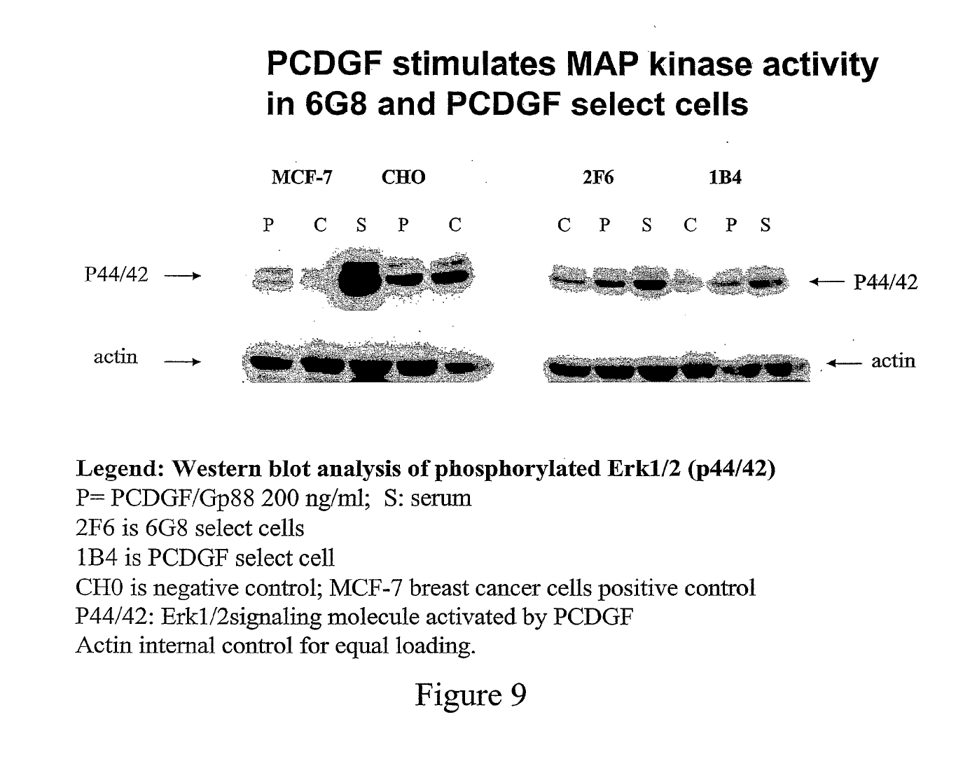

As shown in FIG. 9, 2F6 cells, expression cloned by 6G8 selection, bind to the 6G8 antibody and respond to PCDGF as indicated by stimulating MAP kinase activity. Cell line 1B4 (expression cloned CHO cells) was selected by its ability to bind biotinylated PCDGF. Cells were treated with PCDGF (200 ng/ml), 1% FBS or vehicle only (control C) for 10 minutes prior to preparing cell lysates to examine phosphorylation of erk1/2 MAP kinase using western blot analysis with a ERK1/2 phospho specific antibody. FIG. 9 shows that in both 2F6 and 1 B4 cells, addition of PCDGF/PCDGF leads to increase phosphorylation of Erk1/2. Original CHO cells that do not bind PCDGF do not show any increase p-Erk1/2 in response to PCDGF. Positive control MCF-7 cells show increased phosphorylation in response to both PCDGF and serum.

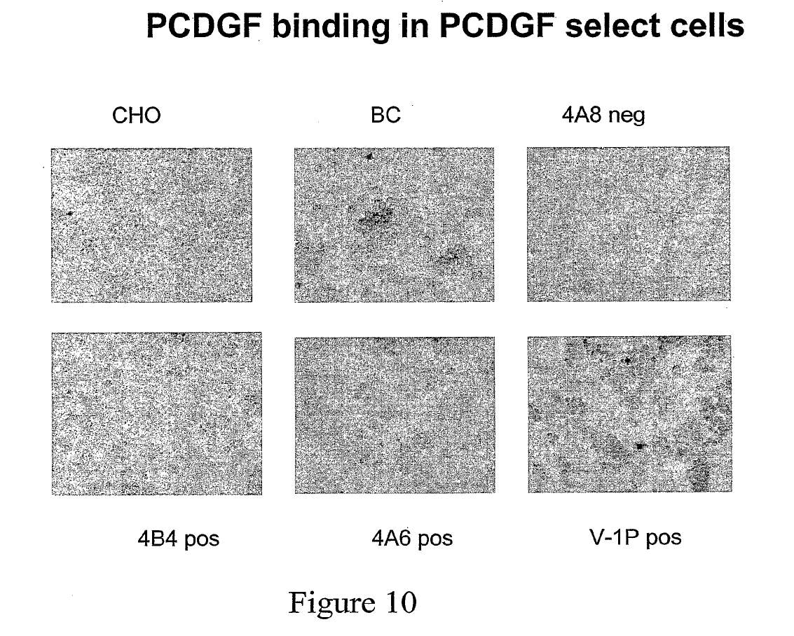

FIG. 10 shows the binding of biotinylated PCDGF to expression cloned cells 4 B4, V-1P. The binding is visualized by binding streptavidin HRP to biotinylated PCDGF/PCDGF bound to cell surface PCDGF receptor followed by chemical staining. Cells that bind PCDGF appear blue. Negative control are CHO cells. FIG. 10 shows the binding of PCDGF to various cell lines including CHO cells (negative control), BC (expressing the PCDGF receptor), 4A8 (negative for PCDGF binding), 4B4 (positive for PCDGF binding), 4A6 (positive for PCDGF binding), and V-1P (positive for PCDGF binding).

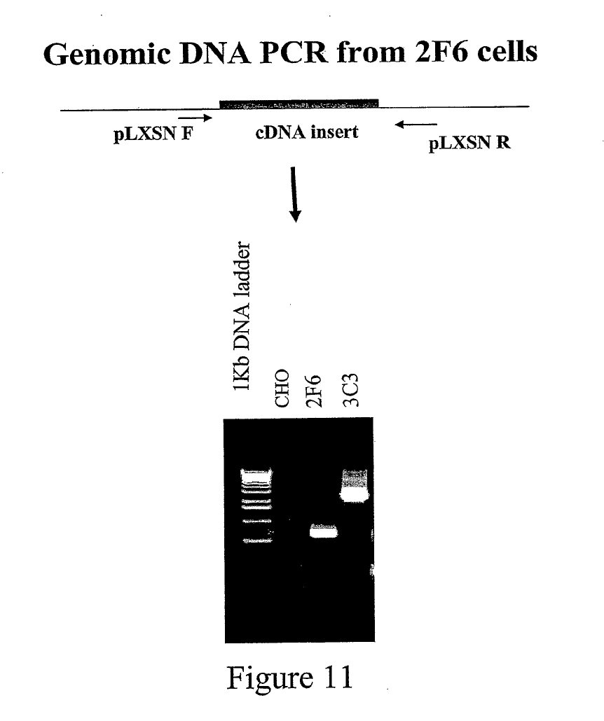

FIG. 11 shows the isolation of genomic DNA from 2F6. A cDNA insert contained in the 6G8 select cells 2F6 genomic DNA was isolated by genomic PCR using retroviral pLXSN primers flanking the cDNA. CHO cells served as a negative control and did bind 6G8 or PCDGF. 3C3 is a negative control, CHO-derived clone that does not bind 6G8 for the PCR procedure.

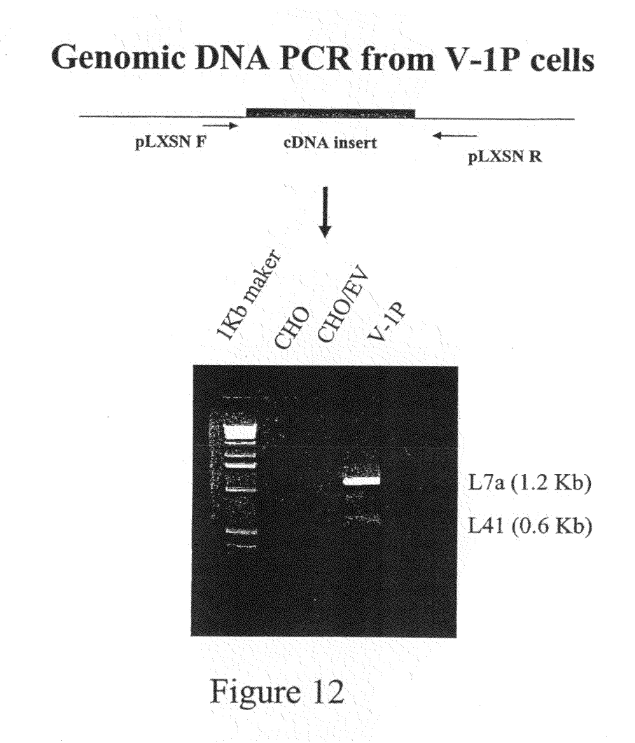

FIG. 12 shows the isolation of a cDNA insert contained in the PCDGF select cells V-1P genomic DNA using retroviral pLXSN primers to amplify the flanking cDNA.

1. A method for diagnosing tumorigenicity comprising: measuring the level of PCDGF receptor expression in a tissue sample suspected of being tumorigenic wherein the presence of PCDGF receptor indicates tumorigenicity.

2. The method of claim 1, further comprising determining whether tumorigenic cells will be responsive to an anti-PCDGF therapy by determining whether there is a measured level of PCDGF receptor in the tissue sample suspected of being tumorigenic.

3. The method of claim 1, wherein the step of measuring the level of PCDGF receptor expression further comprises measuring the level of an isolated nucleic acid molecule consisting essentially of SEQ ID NO: 1 and fragments thereof.

4. The method of claim 1, wherein the step of measuring the level of PCDGF receptor expression comprises measuring the level of an isolated nucleic acid molecule consisting essentially of SEQ ID NO: 2 and fragments thereof.

5. A method for treating a subject having a disease related to the amplification or overexpression of PCDGF, the method comprising, administering a PCDGF receptor or fragment thereof to a patient in amounts effective to inhibit the biological activity of PCDGF.

6. The method of claim 5, wherein the step of administering a PCDGF receptor or fragment thereof comprises administering a protein encoded by an isolated nucleic acid molecule comprising SEQ ID NO: 1 or fragments thereof.

7. The method of claim 5, wherein the step of administering a PCDGF receptor or fragment thereof comprises administering a protein encoded by an isolated nucleic acid molecule consisting essentially of SEQ ID NO: 1 or fragments thereof.

8. The method of claim 5, wherein the step of administering a PCDGF receptor or fragment thereof comprises administering a protein encoded by an isolated nucleic acid molecule comprising SEQ ID NO: 2 or fragments thereof.

9. The method of claim 5, wherein the step of administering a PCDGF receptor or fragment thereof comprises administering a protein encoded by an isolated nucleic acid molecule consisting essentially of SEQ ID NO: 1 or fragments thereof.

10. A method for treating a subject having a disease related to the amplification or overexpression of PCDGF, the method comprising
administering a PCDGF receptor antagonist to a patient in amounts effective to inhibit or interfere with biological activity of PCDGF.

11. The method of claim 10, wherein the PCDGF receptor antagonist is selected from the group consisting of an anti-PCDGF receptor antibody, an anti-PCDGF receptor peptide, an anti-PCDGF receptor antisense nucleic acid molecule, and an anti-PCDGF receptor small inhibitory RNA molecule.

12. The method of claim 11, wherein the PCDGF receptor antibody is produced from a hybridoma cell line selected from the group consisting of ATCC Accession Number PTA-5263 and ATCC Accession Number PTA-5594.

13. An isolated nucleic acid molecule consisting essentially of SEQ ID NO:1 and fragments thereof.

14. An isolated nucleic acid molecule consisting essentially of SEQ ID NO:2 and fragments thereof.

15. An isolated protein encoded by the nucleic acid molecule of claim 13.

16. An isolated protein encoded by the nucleic acid molecule of claim 14.

17. An isolated nucleic acid molecule comprising SEQ ID NO:1 and fragments thereof.

18. An isolated nucleic acid molecule comprising SEQ ID NO:2 and fragments thereof.

19. An isolated protein encoded by the nucleic acid molecule of claim 17.

20. An isolated protein encoded by the nucleic acid molecule of claim 20.
